(19) United States
(12) Patent Application Publication
(10) Pub. No.: US 2012/0041310 A1
(43) Pub. Date: Feb. 16, 2012

(54) Apparatus, System and Method for Ultrasound Powered Neurotelemetry

(75) Inventor: Bruce C. Towe, Mesa, AZ (US)

(73) Assignee: Arizona Board of Regents for and on Behalf of Arizona State University, Scottsdale, AZ (US)

(21) Appl. No.: 13/061,860

(22) PCT Filed: Sep. 1, 2009
(86) PCT No.: PCT/US09/55594

§ 371 Date: Aug. 23, 2011

Related U.S. Application Data
(60) Provisional application No. 61/093,546, filed on Sep. 2, 2008.

(51) Int. Cl. A61B 8/00

(52) U.S. Cl. 600/437

(57) ABSTRACT

The present embodiments provide an apparatus, system, and method for ultrasound powered neurotelemetry. In one embodiment, the apparatus includes a piezoelectric element configured to receive an ultrasonic pulse and convert the electronic pulse into an electric potential. A diode may be coupled to the piezoelectric element, the diode configured to cause an electric current to flow in response to the electric potential. The apparatus may additionally include a reference electrode and a stimulating electrode coupled to the diode. The reference electrode may sense bioelectric activity in a region of body tissue located in proximity to the reference diode. The stimulating electrode may emit a carrier signal, wherein the carrier signal is modulated in response to the bioelectric activity sensed by the reference electrode.

### STATEMENT REGARDING FEDERALLY SPONSORED RESEARCH OR DEVELOPMENT

This invention was made in part with government support under Grant No. 5R21NS063213-01 awarded by the National Institute of Health. The United States Government has certain rights in the invention.

### BACKGROUND

1. Technical Field

The present embodiments relate generally to biomedical engineering and, more particularly, to an apparatus, system, and method for ultrasound powered neurotelemetry.

2. Description of Related Art

Recording of bioelectrical event from the brain, spine, and nervous system in a wireless and minimally invasive manner is an important capability that has received much attention by the National Institute of Health (NIH) in recent years. Investigations of the neural system of the body have been made possible by modern electrophysiological tools. However, such tools have been fundamentally limited with respect to therapeutic uses that go beyond mere research because such devices typically require wires to communicate information. Wires are not desirable and can be sites of infection, mechanical failure, and present dangers of being scraped by abrasion or caught and torn by clothing or environmental objects. Effective biotelemetry obviates the need to pass neural carrier signals through wired connectors on the skin or skull.

There have been some advances in miniature telemetry applications for bioelectrical recording, mostly including batteries or inductive power coupling. There is wide recognition that batteries are undesirable in wireless implant applications and that powering techniques must be by other techniques such as Radio Frequency (RF) induction. Heetderks (1988) performed some early work that examined the limitations on inductive power coupling between two separated loop antennas, one external and one internal to the body at various frequencies up to 20 MHz. There are some fundamental limitations on this process relative to the needed and relatively large size of the implanted antenna size for at-depth applications.

Sophisticated analog and more recently digital circuitry mated to wireless telemetry have been reported for neuroprostheses. A review of this activity has been conducted by Wise et al. (2004). Present methods of achieving multichannel wireless interfaces involve silicon VSLI circuitry and are relatively complex devices involving arrays of high performance bioamplifiers, multiplexers, and wideband RF communication. These devices tend to have thermal dissipation problems, and supplying power to neuroprosthetics becomes a major issue.

The use of passive RF circuitry for biotelemetry has a long history. These devices typically use changes in mutual inductance or reflected impedance between two resonant circuits. Passive techniques have the advantage of low power needs and the potential for reduced dependency on RF power induction for active circuitry. In 1986 Towe (1986) demonstrated a low power quasi-passive technique of resonant frequency shifting to telemeter analog bioelectrical waveforms on a subcarrier. The NIH has supported the development of passive biotelemetry devices at the WIMS center at the University of Michigan (http://www.wimserc.org/). There have been reports by Najafi, Wise, and others at Michigan (Harpster et al., 2002; Takahata et al., 2003) of passive telemetry applications for humidity, for stents, as well as for parameters such as pressure (DeHennis et al., 2002).

Recently Towe (2007) presented a method to considerably reduce the complexity of passive telemetry by exploiting the unique properties of semiconductor RF varactor diodes. This wireless biotelemetry system is similar to the technology of RF-ID tags and presents an RF backscatter method to telemeter low level bioelectric events over short distances, without the use of integrated bioamplifiers or conventional transmitters. The approach employs the voltage-variable capacitance function of varactor diodes to allow biopotentials to directly alter the tuning of an Inductive/Capacitive (LC) resonant circuit. The tradeoff is the need for relatively more complex synchronous carrier demodulation schemes external to the body.

The human body can be electrically modeled as a volume conducting medium. Natural or artificial current sources in the interior of the body will thereby produce skin surface potentials. This principle has been used for biotelemetry by Mingui et al. (2003) and Linsey et al. (1998) by using implanted amplifiers connected to sensors or biopotential electrodes and then driving relatively higher local currents in tissues to cause large signals at the skin surface. Difficulties include the achievement of multichannel operation, the relatively large bulk of devices reported so far, and the need for induced power to run the amplifier.

This application incorporates by reference provisional patent application No. 60/916,152 filed on May 4, 2007 in its entirety.

### SUMMARY

Multichannel, totally integrated neuro-recording by ultra-miniature wireless systems is a long-sought goal in neuroengineering. It would allow us to achieve multiple simultaneous recordings of bioelectrical events such as to constitute a map of the activity at multiple sites. Mapping would allow a more complete understanding of ensembles of activity that are further apart than a few millimeters of each other and so useful to record from multiple sites in the brain such as motor and sensory centers spine, or nervous system.

Ultraminiature wireless bioelectric monitoring tools could be useful and important in design of neuroprosthetics medical rehabilitation, diagnostics, therapeutics, and to the relatively new field of man-machine interfaces. It is widely recognized that microminiature wireless interfaces to the body interior would enhance the development of advanced neural interfaces leading to prostheses in many forms.

The present embodiments provide an apparatus, system, and method for ultrasound powered neurotelemetry. In one embodiment, the apparatus includes a piezoelectric element configured to receive an ultrasonic pulse and convert the electronic pulse into an electric potential. A diode may be coupled to the piezoelectric element, the diode configured to cause an electric current to flow in response to the electric potential. The apparatus may additionally include a reference electrode and a stimulating electrode coupled to the diode. The reference electrode may sense bioelectric activity in a region of body tissue located in proximity to the reference diode. The stimulating electrode may emit a carrier signal, wherein the carrier signal is modulated in response to the bioelectric activity sensed by the reference electrode.

In a further embodiment, the apparatus may include a housing configured to house the piezoelectric element and the diode. The housing may also house at least a portion of both the reference electrode and the stimulating electrode. The housing may reduce potential infection due to immune system response to the apparatus.

In one embodiment, the diode may be a semiconductive mixer diode. The reference electrode may be coupled to a cathode portion of the diode and the stimulating electrode may be coupled to an anode portion of the diode. Additionally, the piezoelectric element may apply an electric potential to the diode that is slightly below the threshold voltage of the diode. In another embodiment, the diode may be zero-potential biased. In a further embodiment, the diode may be further configured to mix a bioelectric signal generated by bioelectric activity sensed by the reference electrode with the carrier signal.

An alternative embodiment of an apparatus is also presented. In this embodiment, the apparatus may include a biopotential electrode configured to detect a carrier signal on a skin surface. The apparatus may also include an amplifier coupled to the biopotential electrode, the amplifier configured to amplify the carrier signal across a predetermined frequency range. The apparatus may further include a range gate circuit coupled to the amplifier, the range gate circuit configured to capture the carrier signal within a specified time range. In a further embodiment, the apparatus may include a sample and hold circuit coupled to the range gate circuit, the sample and hold circuit configured to construct a waveform associated with the carrier signal. Additionally, the apparatus may include a bandpass filter coupled to the sample and hold circuit, the bandpass filter configured to smooth the waveform. The apparatus may also include a waveform output device coupled to the bandpass filter, the waveform output device configured to produce a waveform display.

A system in accordance with the present embodiments is also presented, the system including an ultrasound source configured to generate an ultrasound pulse, an implant configured to be implanted in body tissue, and a receiver configured to detect the carrier signal. The implant may include a piezoelectric element configured to receive an ultrasonic pulse and convert the electronic pulse into an electric potential, a diode coupled to the piezoelectric element, the diode configured to cause an electric current to flow in response to the electric potential, a reference electrode coupled to the diode, the reference electrode configured to sense bioelectric activity in a region of the body tissue located in proximity to the reference diode, and a stimulating electrode coupled to the diode, the stimulating diode configured to emit an carrier signal, wherein the carrier signal is modulated in response to the bioelectric activity sensed by the reference electrode.

A method is also presented in accordance with the present embodiments. In one embodiment, the method includes receiving an ultrasound pulse, converting the ultrasound pulse into an electric potential, causing an electric current to flow through a diode from a reference electrode to a stimulating electrode in response to the electric potential, and emitting an carrier signal from the stimulating electrode, wherein the carrier signal is modulated in response to bioelectric activity in a region of body tissue located in proximity to the reference electrode.

A further embodiment of the method may include detecting the carrier signal on a skin surface, amplifying the carrier signal across a predetermined frequency range, capturing the carrier signal within a specified time range, constructing a waveform associated with the carrier signal, smoothing the waveform, and producing a waveform display.

The term “coupled” is defined as connected, although not necessarily directly, and not necessarily mechanically. The terms “a” and “an” are defined as one or more unless this disclosure explicitly requires otherwise. The terms “substantially,” “approximately,” “about,” and variations thereof are defined as being largely but not necessarily wholly what is specified, as understood by a person of ordinary skill in the art. In one non-limiting embodiment, the term substantially refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but it may also be configured in ways other than those specifically described herein.

### BRIEF DESCRIPTION OF THE SEVERAL VIEWS OF THE DRAWINGS

For a more complete understanding of the present embodiments, reference is now made to the following drawings, in which:

FIG. 1 is a schematic block diagram illustrating one embodiment of a system for ultrasound powered neurotelemetry;

FIG. 2 is a schematic block diagram illustrating another embodiment of a system for ultrasound powered neurotelemetry;

FIGS. 3A-3C are schematic diagrams illustrating various embodiments of an implant for ultrasound powered neurotelemetry;

FIG. 4 is a schematic block diagram illustrating one embodiment of a receiver;

FIG. 5 is a schematic flowchart diagram illustrating one embodiment of a method for ultrasound powered neurotelemetry;

FIG. 6A is a graph illustrating a voltage response of a diode in accordance with the present embodiments;

FIG. 6B illustrates a response of a piezoelectric element in response to an ultrasound pulse;

FIG. 7A is a frequency measurement of an unmodulated carrier in accordance with the present embodiments;

FIG. 7B is a frequency measurement of a modulated carrier in accordance with the present embodiments;

FIG. 8 is a graph of a voltage level as a function of depth of placement of the implant; and

FIG. 9 is an illustration of one embodiment of an implant with size comparison reference objects.

### DETAILED DESCRIPTION OF THE DRAWINGS

In the following detailed description, reference is made to the accompanying drawings that illustrate embodiments of the present invention. These embodiments are described in sufficient detail to enable a person of ordinary skill in the art to practice the invention without undue experimentation. It should be understood, however, that the embodiments and examples described herein are given by way of illustration only, and not by way of limitation. Various substitutions, modifications, additions, and rearrangements may be made without departing from the spirit of the present invention. Therefore, the description that follows is not to be taken in a limited sense, and the scope of the present invention is defined only by the appended claims.

Bioelectrical currents flowing in excitable tissue in the body may be modeled as current sources in the range of tens to hundreds of microamperes and with associated electric fields in the range of microvolts to tens of millivolts in the case of transmembrane potentials. These devices can be understood from volume conductor propagation of a small dipolar current source in tissue that follows well understood rules. The potential V appears on the skin surface as:

V=id cos θ/4πσr²

where i is the current flow over a dipole length d, σ is the medium conductivity, and r is the distance from the center of the dipole to the skin surface. Thus there is a square law loss of the signal strength generated by the current source at depth from the body surface and there is a vector relationship to orientation of the electrode pairs.

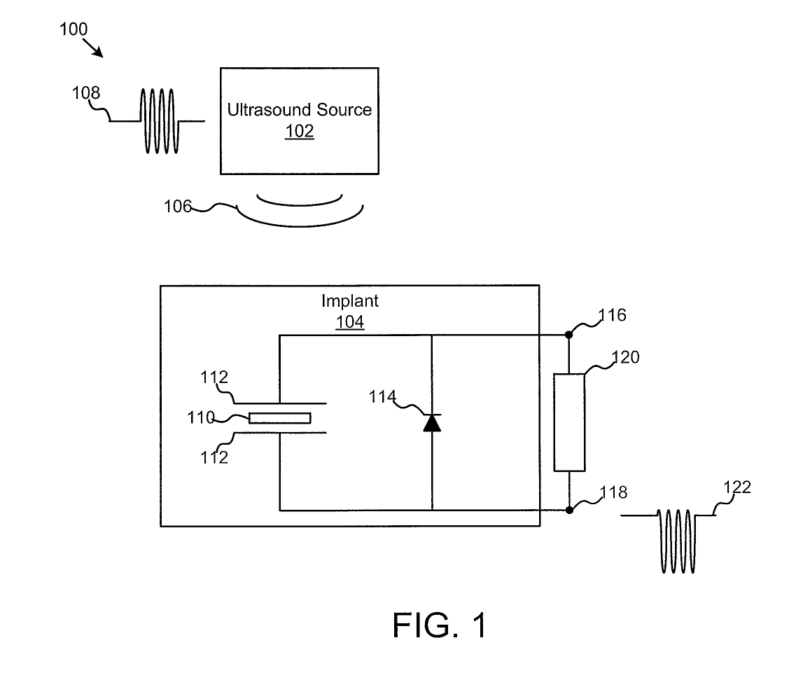

In the system 100 illustrated in FIG. 1, the bioelectrical event waveforms are relayed to the skin for detection by a small implant 104 device that senses local events and then modulates them on an electrical carrier for remote detection at the body surface.

The characteristics of p-n junction diodes, such as those that may be suitable for diode 114, can be substantially varied in their characteristics by biopotentials when reverse biased or when biased near their turn-on threshold. Parameters such as junction capacitance, effective resistance, and nonlinear second harmonic production can all be substantially affected by submillivolt level electrical signals applied to them. This process can be conceived as the diode acting as a (nonlinear) multiplying element. The Shockley equation shows the relationship of the diode forward current to an applied bias voltage.

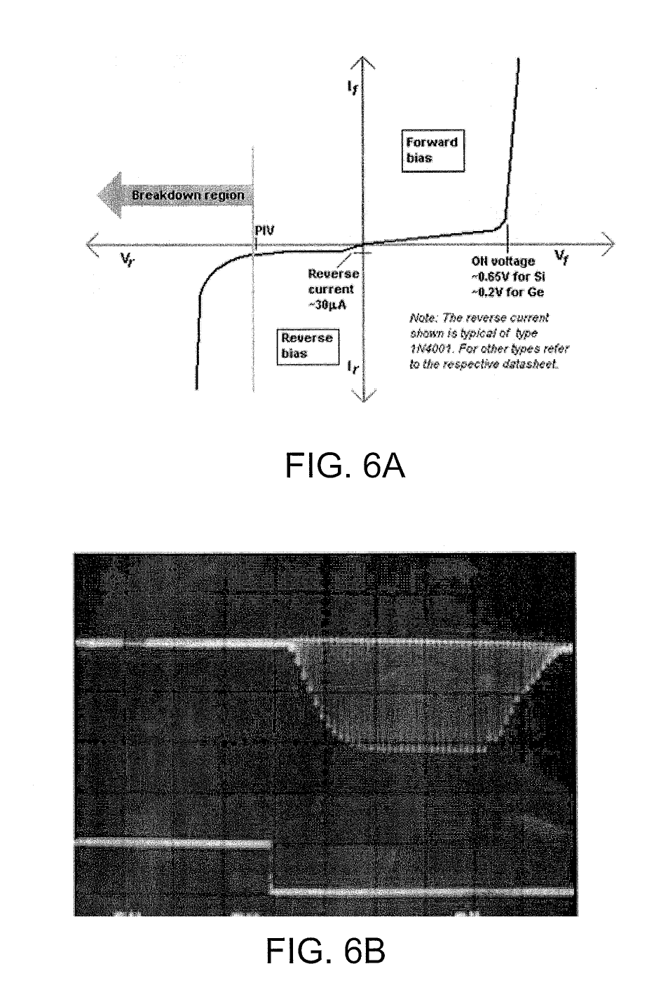

I=I_{S}(e^{V}^{_{D}}^{/(nV}^{_{T}}⁾−1)

where I is the diode current, Iₛ is a scale factor called the saturation current, V_{D} is the voltage across the diode, V_{T} is the thermal voltage, and n is emission coefficient. FIG. 6A shows the sharp knee in the i-v curve near threshold. By operating V_{D} slightly below this point (which moves towards the origin in zero-bias type Shottky diodes) millivolt biopotential signals may amplitude modulate an externally applied and relatively high frequency carrier current also passing through the diode. This process is known as mixing or sometimes as intermodulation when applied to the design of radio devices. This process may be accomplished using high performance low-noise mixer diodes 114, such as those used in RF communications, at microvolt signal levels. Accordingly, in such an embodiment, the mixing process may not be a significant source of noise or limitation on the biopotential intermodulation process.

In one embodiment, a high frequency (megahertz) carrier current signal may be applied to the diode 114 from a small attached piezoelectric element 110. The piezoelectric element 110 may include a polymer material (PVDF). Alternatively, the piezoelectric element 110 may include a crystalline and ceramic materials such as quartz, barium titanate, lead zirconium titanate (PZT), or the like. The piezoelectric element 110 may be driven to generate an oscillating current through the diode 114 by an ultrasound wave or pulse. FIG. 1 illustrates one embodiment of an electrical circuit configuration where the impedance of the tissue volume conductivity 120 is in parallel with the mixer diode 114 and piezoelectric element 110.

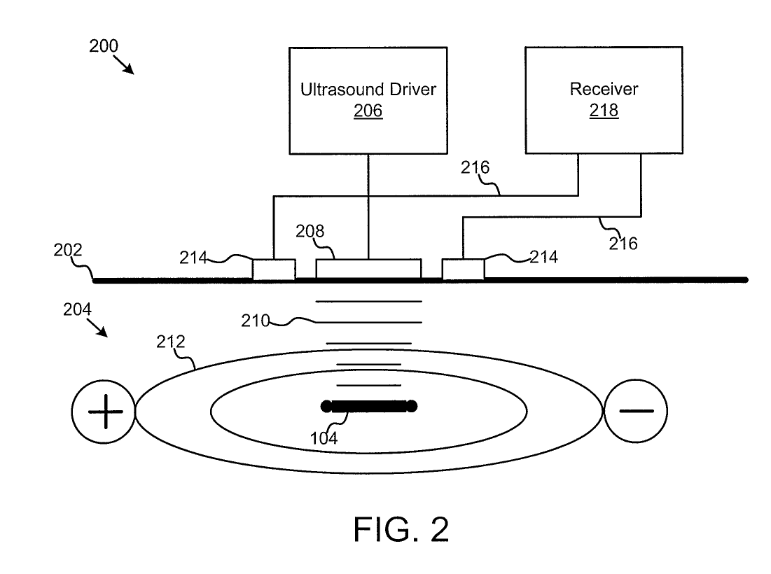

The carrier current through the diode 114 may be amplitude modulated by a lower frequency (0-10 kHz) signal from local microelectrodes 116-118. When placed in tissue, volume conductivity carries the biopotential modulated carrier current to the surface where it is detected by a second set of surface bioelectrodes (illustrated as elements 214 in FIG. 2). Demodulation of the detected signal reproduces the original biopotential waveform.

In such an embodiment, the implant 104 assembly may intermodulate a bioelectrical event on a superimposed high frequency carrier whose energy is obtained piezoelectrically from an ultrasound frequency pressure wave. Over a small change in biopotential, characterized by the impedance value 120, the changes in the carrier current through the diode 114 may be reasonably linear. At low drive levels, the diode 114 may present a relatively high source impedance to the electrodes 214 which, according to system tests, appears to work satisfactorily.

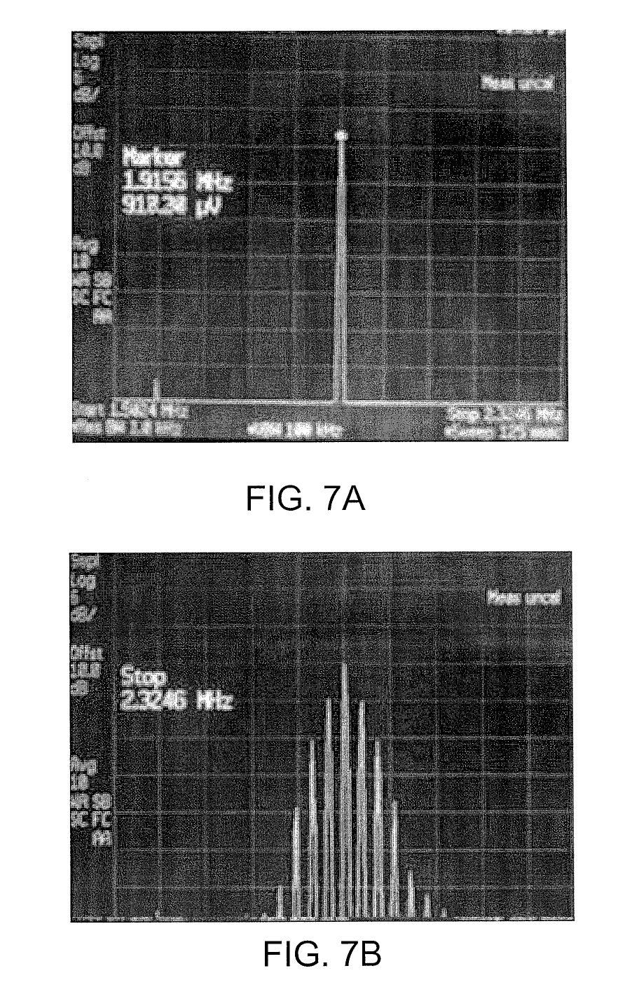

FIG. 7A shows a spectrum analyzer output when connected to skin surface electrodes 214. The implant 104 rectifies the 1 MHz ultrasound to the spike seen. FIG. 7B shows the effect of driving simulated bioelectric activity in a test setup at microamperes and a frequency of 30 kHz using two small silver electrodes immersed in a fluid tank in a region in proximity to the reference electrode 116 and the stimulating electrode 118. The spread spectrum with multiple sideband spikes in addition to the main carrier is a clear indication of amplitude intermodulation of the volume conducted current with the carrier current generated by the piezoelectric element 110 in response to the ultrasound beam 108.

Preliminary test data shows a strong intermodulation effect that is exhibited in currents 112 in volume conductors. Effectively, the volume current captures the bioelectrical event of interest. Remote detection and demodulation of the surface-detected carrier 112 may reproduce the bioelectric event waveform.

In one embodiment, the intermodulation effect may be relatively frequency independent with modern RF diodes 114, and at least extends over the range from dc to tens of MHz with typical diodes 114. This easily encompasses the ultrasound and bioelectrical frequency ranges.

For example, the ultrasound source 102 may include an ultrasound transducer. The transducer may generate a variable power ultrasound pulse 106 at a frequency range of about 400 kHz to 5 MHz. The ultrasound pulse power may be varied in order to provide consistent power levels to the implant at varying depths in tissue. The ultrasound pulse 106 may generate sound pressure waves that pass through the skin and other tissue in a body. In one embodiment, the ultrasound source 102 may generate the ultrasound pulse 106 in response to a control signal 108. The power level and/or frequency of the ultrasound pulse 106 may be determined by a combination of the control signal 108 properties and the characteristics of the ultrasound source 102.

In one embodiment, the piezoelectric element 110 may receive an incident ultrasound pulse 106. The piezoelectric element 110 may convert the mechanical pressure of the incident ultrasound pulse 106 into electrical power. The electrical power generated by the piezoelectric element 110 may be conducted by one or more conductive plates 112 coupled to the piezoelectric element 110. The diode 114 may be coupled in electric parallel to the piezoelectric element 110 through the conductive plates 112.

In one embodiment, a reference electrode 116 may be coupled to a cathode portion of the diode 114. Additionally, a stimulating electrode 118 may be coupled to an anode portion of the diode 114. In a further embodiment, the implant 104 may be placed in a body, and the reference electrode 116 and the stimulating electrode 118 may be placed in contact with a portion of body tissue. The impedance of the body tissue is represented by an equivalent impedance value 120.

An electric potential may be generated by the piezoelectric element 110 in response to the incident ultrasound pulse 106. The level of electric potential applied to the diode 114 may put the diode 114 in a state that is near its threshold value. Additionally, the electric potential may cause a current generated by the piezoelectric element 110 to flow from the reference electrode 116, through the diode 114, to the stimulating electrode 118. In this embodiment, the current may have a frequency of around 400 kHz to 5 MHz. For example, the diode 114 may conduct a 2 MHz carrier current in response to the electric potential applied by the piezoelectric element 110. FIG. 6B illustrates one embodiment of a carrier current generated by a piezoelectric element 110 in response to illumination by an ultrasound beam 108 with a frequency of 1 MHz and a power of 10 W/cm². FIG. 6 B illustrates that the carrier current may also have a frequency of 1 MHz. Indeed, the carrier current may have the same frequency as the frequency of the incident ultrasound pulse 108.

In such an embodiment, the diode 114 may mix or intermodulate bioelectric activity occurring in the proximity of the electrodes 116, 118 with the carrier current. In one embodiment, the carrier current may be amplitude modulated by the bioelectric signal detected by the local electrodes 116-118 at a frequency between 0-10 kHz. The modulated carrier current may then be transmitted as a modulated carrier signal 122 through volume conduction to the skin.

FIG. 2 illustrates another embodiment of a system 200 for ultrasound powered neurotelemetry. As depicted, the ultrasound source 102 is replaced by a ultrasound driver 206 coupled to a transducer 208. The transducer 208 may be placed in contact with the skin 202. An implant 104 may be placed under the skin 202 within body tissue 204. For example, the implant 104 may be placed in brain tissue, heart tissue, or other body tissues. The ultrasound driver 206 may generate a driving signal causing the transducer 208 to emit an ultrasound pulse 210 through the skin 202 into the tissue 204. The implant 104 may receive the ultrasound pulse 210 and emit a modulated carrier signal 212 through a volume conduction of electrical field lines back to the skin.

One or more electrodes 214 may be in electrical contact with the skin 202. The electrodes 214 may detect the modulated signal 212 and transmit the modulated signal 212 over a wired connection 216 to a receiver 218. Alternatively, the electrodes 214 may communicate the signal 212 to the receiver 218 over a wireless RF link (not shown). The receiver 218 may demodulate the signal 212 to obtain information about the bioelectric activity sensed by the implant 104. The receiver 218 may use amplitude and/or phase demodulation to decode the bioelectrical event signal. Advantageously, such a system 200 may be implanted directly in the tissue 204 without the need for internally coupled lead wires or bulky open-loop inductive components.

In one embodiment, the receiver 218 may provide single-channel demodulation for a single implant 104. This implant 104 design approach can operate in at least two different modes but in each case it drives a high frequency carrier wave 212 in tissue 204 containing a volume current driven by an additional set of electrodes that mimic a bioelectrical current.

The highest system sensitivity to low level bioelectric events can be achieved by driving the implant 104 with a continuous-duty ultrasound beam 210 such as comparatively might be used in medical Doppler flow or similar applications. For single implant 104, or in situations where implants 104 are spaced such that individual ultrasound beams 210 may be directed at individual implants 104 without overlap, the demodulation process that recovers the biopotential waveform from the surface detected carrier wave performed by the receiver 218 may be relatively straightforward.

For example, commercially available high frequency lock-in amplifiers may be used for demodulation directly from surface electrodes 214. In such an embodiment, digital outputs of the lock-in amplifier may be recorded by a computer configured to make plots and tables of the data.

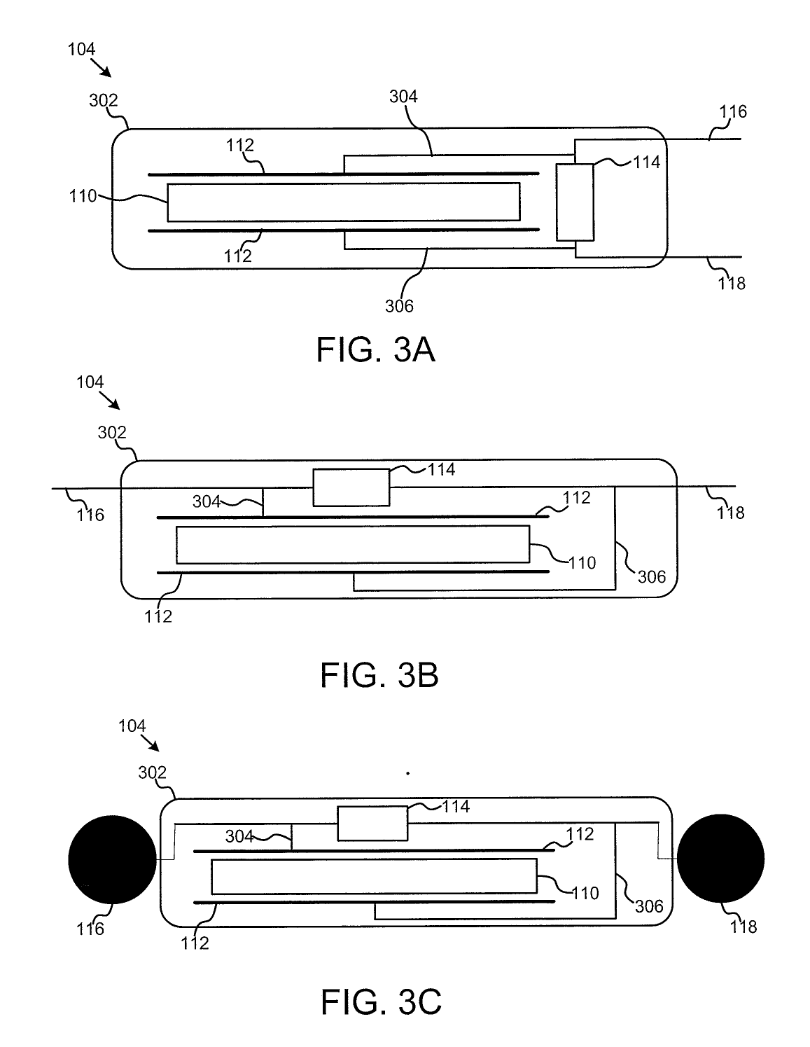

FIGS. 3A-3C illustrate various embodiments of an implant 104. In FIG. 3A, the implant 104 includes an elongated piezoelectric element 110 coupled to two conductive plates 112. This example also includes a semiconductor diode 114 coupled in electric parallel to the piezoelectric element 110 by electrical coupling lines 304, 306. These lines may be soldered to the conductive plates 112 and the semiconductor diode 114. Alternatively, the coupling lines 304, 306 may be deposited through physical deposition or chemical deposition processes. In another embodiment, the coupling lines 304, 306 may be coupled to the conductive plates 112 using silver epoxy or by hot pressing a silver coating. The diode 114 may also be coupled to a reference electrode 116 and a stimulating electrode 118. In one embodiment, these electrodes 116, 118 may protrude through a protective coating or housing 302 which houses the other elements of the implant. The housing may protect the components from corrosion and may reduce infection resulting from immune system reactions to the components.

In one specific example, the implant 104 may be constructed using a commercial quality PVDF plastic and a packaged Shottky diode. In a further embodiment, the piezoelectric current response of the PVDF may be increased by stacking thin sheets of approximately 25 micrometer thickness in electric parallel. The overall thickness of the piezoelectric element, including bonding thicknesses, may be around 250-350 micrometers, and form a solid structure. The piezoelectric element may be cut into various sizes depending on power requirements. For example, the piezoelectric element may have a width-height measurement of 0.8 mm×2 mm, 1.5 mm×3 mm, 2.5 mm×5 mm, or the like.

The diode 114 may comprise an ultraminiature surface mount diode, such as an SOT-363 package, having an epoxy overcoat. Indeed the size of these packages may be reduced, by sanding the package with light grit sand paper, to a thickness of between 0.6 mm and 0.9 mm. In such an embodiment, the piezoelectric element 110 and the diode 114 may be sized to fit through the lumen of a #16 gauge syringe needle.

In the embodiment depicted in FIG. 3A, the reference electrode 116 and the stimulating electrode 118 both protrude from the housing 302 at the same end. The electrodes 116, 118 may protrude from the housing 302 by about millimeter. In various embodiments, the electrodes 116, 118 may protrude more or less depending on the particular bioelectrical characteristics of the tissue in which the implant 104 is placed.

FIG. 3B illustrates an alternative embodiment of the implant 104 in which the reference electrode 116 is positioned on a first end of the implant 104 and the stimulating electrode 118 is positioned on a second end of the implant 104.

FIG. 3C illustrates yet another embodiment of the implant 104 in which the electrodes 116, 118 are substantially spherical or ball shaped. The spherical electrodes 116, 118 may have a diameter of approximately 0.9 mm. In one embodiment, the spherical electrodes 116, 118 may be formed of silver chloride. The silver chloride balls may be formed in a flame by melting silver wire and then attached to the body of the implant 104 using silver bearing epoxy and held in place using UltraViolet (UV) curing epoxy. The silver-chloride balls may be chlorided through exposure to saline.

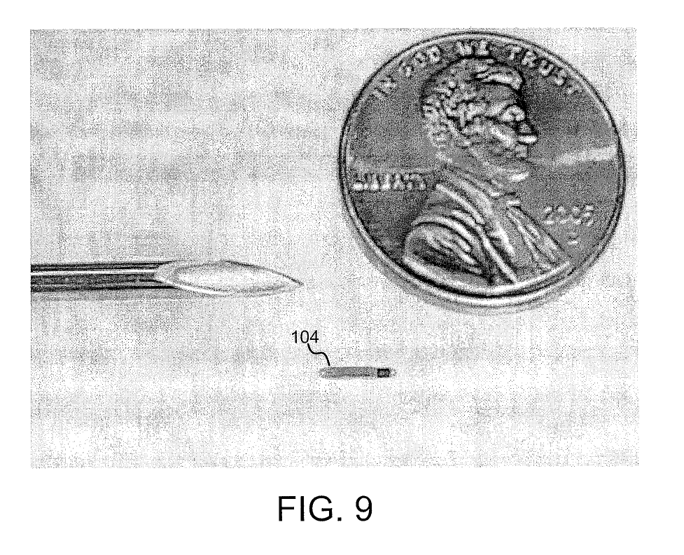

FIG. 9 illustrates a size comparison of one embodiment of an implant 104. In the depicted embodiment, the implant 104 may be sized to pass through the lumen of a syringe needle, such as a #16 gauge needle.

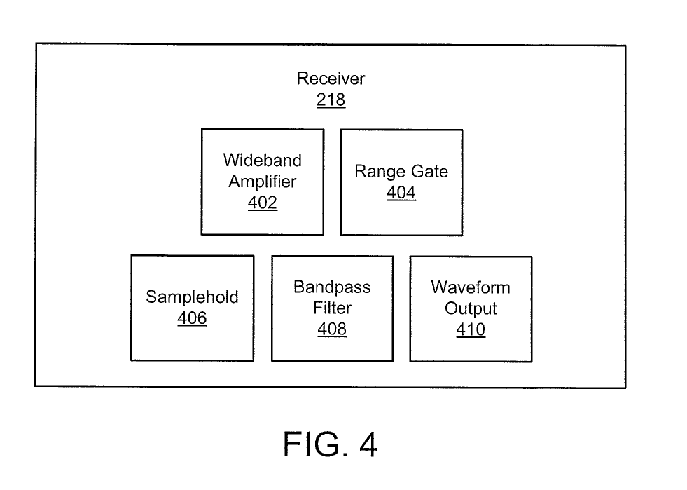

FIG. 4 illustrates a further embodiment of a receiver 208. In the depicted embodiment, the receiver 208 may include a wideband amplifier 402, a range-gate circuit 404, a samplehold circuit 406, a bandpass filter 408, and a waveform output 410.

One advantage of a telemetry approach using ultrasound is that it permits time-serial excitation and readout of multiple implants 104. Advantageously, there is no added complexity on the implants 104 to achieve this. In one embodiment, multichannel operation may be accomplished by simply placing additional implants 104 within the path of the incident ultrasound beam 210.

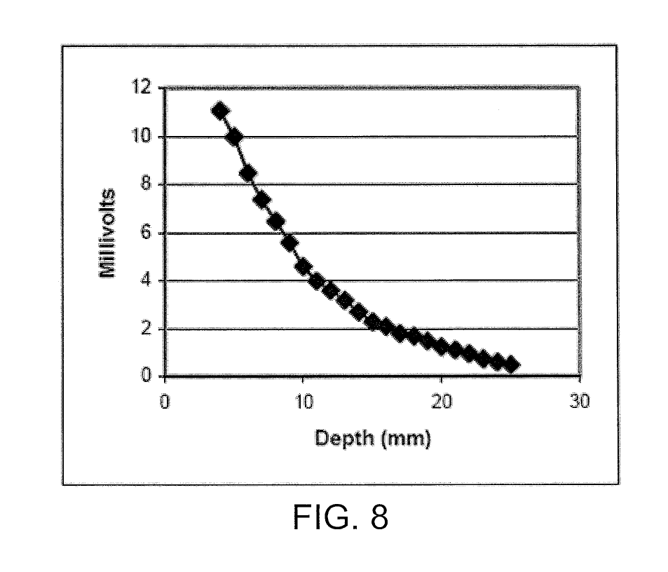

The wideband amplifier 402 may be required to amplified low level carrier signals 112 detected by the surface electrodes 214. Amplification may be particularly useful as the depth of the implant 104 placement increases. An additional feature for the wideband amplifier 402 may be a low noise contribution level. As shown in FIG. 8, the body tissue 2014 may significantly attenuate the carrier signal 112 as the volume conduction current carries it to the skin surface from various depths. FIG. 8 shows that at depths of 10 mm and more, the surface electrodes 214 may only detect 4 millivolts or less of the carrier signal 112. Thus, the wideband amplifier 402 may include a high degree of noise isolation in order to provide a sufficient signal to noise ratio (SNR).

Pulsed ultrasound drivers 206 employed for multichannel applications may require a more complex process of demodulation by the receiver. For example, transit time range gating by a range-gate circuit 404 may separate the signals from multiple implants 104 along a beam of ultrasound 210.

In one embodiment, an ultrasound pulse 210 may travel at a quantifiable rate through the various body tissues 204. For example, 15 microsecond delay may occur between a transmitted ultrasonic pulse 210 and detected electrical response 112 from a 2.2 cm spacing between the implant 104 and the ultrasound transducer 208. Thus the delay time indicates implant 104 distance from the transducer 208. Such data may be used by the range-gate circuit 404 to separate received carrier signals 112 by time delay and identify corresponding implants 104 based on a correlation between known depth of placement and response timing. For example, the electrical responses 112 of multiple implants 104 placed at various depths along a line can potentially be discriminated from each other by noting delay times and by using an electronic gate to admit signals from only specific depths for further processing.

The electrical design connects the surface biopotential electrodes 214 to a high gain broad bandwidth amplifier 402 to accommodate low level surface potentials whose amplitudes may fall into the tens of microvolt range. The amplified signal may be passed to the range-gate circuit 404. The range gate circuit 404 may comprise a series of electronic gates that pass only electrode signals that are delayed by a selectable windowed interval. The width and timing of the gate opening may define the depth of the implant 104. Additionally, the width and timing of the gate opening may contribute to the system range resolution. This allows directing of the bioelectrically modulated carrier currents to be directed into separate data channel streams.

In one embodiment ultrasound imaging system pulse repetition rates be in the range of 4 kHz to 10 kHz for near surface imaging. This suggests that bioelectrical bandwidths in a pulsed sampling mode could be as much as 2 kHz to 5 kHz under conditions of Nyquist limitation. Ultrasound imaging systems can have millimeter-order spatial resolution which thus suggests an ability to separate signals from closely spaced implants 104.

Each data stream may be reconstructed into continuous waveform data using a sample-hold circuit 406. The sample-hold circuit may be synced to the range gate circuit 404. High speed sampled waveform segments may be connected and smoothed by a low pass filter or a bandpass filter 408. Multiple bioelectric waveforms may be recovered by the receiver 218 with a range resolution that is determined by a multitude of factors including the gate timing width, the transducer frequency and pulse ring-down, the size of the implants 104, and practical constraints on the density of placement of the implants 104. The resulting waveforms may be stored in a database on a computer using an interface to the waveform output 410. Alternatively, the waveforms may be displayed on a graphical display or screen, plotted, or printed using a data driven printing device.

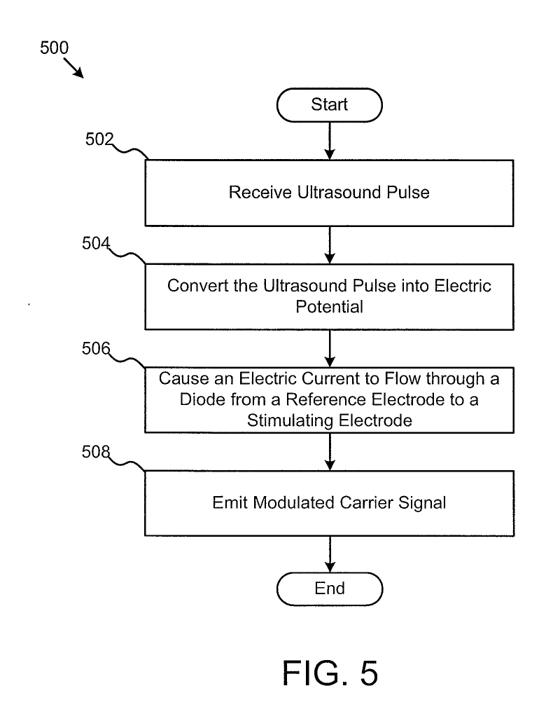

FIG. 5 illustrates one embodiment of a method 500 for ultrasound powered neurotelemetry. In one embodiment, the method 500 starts when the piezoelectric element 110 of the implant 104 receives 502 an ultrasound pulse 108. The ultrasound pulse 108 may be generated by an ultrasound source 102. In one embodiment, the ultrasound source 102 includes a transducer 208 and an ultrasound driver 206. The ultrasound pulse may have a frequency of between 400 kHz and 5 MHz.

The method 500 may continue when the piezoelectric element 110 converts 504 the ultrasound pulse 108 into electrical potential. In a further embodiment, the piezoelectric element may convert the physical pressure power of the ultrasound pulse into sufficient electrical power to supply both the voltage and current needs of the diode 114 circuit.

In a further embodiment, the method 500 may include causing 506 an electric current to flow through the diode 114 from a reference electrode 116 to a stimulating electrode 118 in response to the electric potential generated by the piezoelectric element 110. The piezoelectric element 110 may also supply sufficient current to establish a carrier current through the diode. The carrier current may be modulated through the diode 114 by mixing the carrier current with any bioelectric activity that may occur in proximity of the reference electrode 116. In a further embodiment, the implant 104 may emit 508 the modulated carrier signal 112 into body tissue 204. The modulated carrier signal 112 may be conducted through volume conduction to the skin 202 where it may be detected by one or more surface electrodes 214, and the method 500 ends. In a further embodiment, the surface electrodes 214 may communicate the carrier signal 112 to the receiver 218 for demodulation.

The present method 500 may be performed by multiple implants 104 placed in different regions of the body tissue 204. In a certain embodiment, multiple implants 104 may respond according to the present method 500 in response to a single ultrasound pulse 108. In such an embodiment, the receiver 218 may demodulate and assemble the various carrier signals 112 for analysis using techniques described above with reference to FIG. 4.

Although certain embodiments of the present invention and their advantages have been described herein in detail, it should be understood that various changes, substitutions and alterations can be made without departing from the spirit and scope of the invention as defined by the appended claims. Moreover, the scope of the present invention is not intended to be limited to the particular embodiments of the processes, machines, manufactures, means, methods, and steps described herein. As a person of ordinary skill in the art will readily appreciate from this disclosure, other processes, machines, manufactures, means, methods, or steps, presently existing or later to be developed that perforin substantially the same function or achieve substantially the same result as the corresponding embodiments described herein may be utilized according to the present invention. Accordingly, the appended claims are intended to include within their scope such processes, machines, manufactures, means, methods, or steps.

### REFERENCES

DeHennis et al., In: A Double-Sided Single-Chip Wireless Pressure Sensor, Micro Electro Mech. Sys., 15^{th} IEEE Intl. Conf., 252-255, 2002.
Harpster et al., Sensors Actuators A: Physical, 95(2-3):100-07, 2002.
Heetderks, IEEE Trans. Biomed. Engineering, 35(5):323, 1988.
Lindsey et al., IEEE Trans. Biomed. Engineering, 45(5):614-619, 1998.
Minqui et al., IEEE Trans. Neural Sys. Rehab. Engin., 11(2):189-192, 2003.
Takahata et al., In: Stentenna: A Micromachined Antenna Stent For Wireless Monitoring Of Implantable Microsensors, Eng. Med. Biol. Soc., Proc. 25^{th} Ann. Intl. Conf. IEEE, 4:3360-3363, 2003.
Towe, IEEE Trans. Biomed. Engineering, BME-33:10, 1986.
Towe, In: Passive Backscatter Biotelemetry for Neural Interfacing, 3^{rd} Intl. IEEE/EMBS Conf., 144-147, 2007.
Wise et al., IEEE Trans. Biomed. Engineering, 92(1):76-97, 2004.

1. An apparatus comprising:
a piezoelectric element configured to receive an ultrasonic pulse and convert the electronic pulse into an electric potential;
a diode coupled to the piezoelectric element, the diode configured to cause an electric current to flow in response to the electric potential;
a reference electrode coupled to the diode, the reference electrode configured to sense bioelectric activity in a region of body tissue located in proximity to the reference diode; and
a stimulating electrode coupled to the diode, the stimulating diode configured to emit a carrier signal, wherein the carrier signal is modulated in response to the bioelectric activity sensed by the reference electrode.

2. The apparatus of claim 1, further comprising a housing configured to house the piezoelectric element and the diode.

3. The apparatus of claim 1, wherein the diode further comprises a semiconductive mixer diode.

4. The apparatus of claim 1, wherein the reference electrode is coupled to a cathode portion of the diode and the stimulating electrode is coupled to an anode portion of the diode.

5. The apparatus of claim 1, wherein the piezoelectric element is further configured to apply an electric potential to the diode that is slightly below the threshold voltage of the diode.

6. The apparatus of claim 1, wherein the diode is further configured to mix a bioelectric signal generated by bioelectric activity sensed by the reference electrode with the carrier signal.

7. An apparatus comprising:
a biopotential electrode configured to detect a carrier signal on a skin surface;
an amplifier coupled to the biopotential electrode, the amplifier configured to amplify the carrier signal across a predetermined frequency range;
a range gate circuit coupled to the amplifier, the range gate circuit configured to capture the carrier signal within a specified time range;
a sample and hold circuit coupled to the range gate circuit, the sample and hold circuit configured to construct a waveform associated with the carrier signal;
a bandpass filter coupled to the sample and hold circuit, the bandpass filter configured to smooth the waveform; and
a waveform output device coupled to the bandpass filter, the waveform output device configured to produce a waveform display.

8. A system comprising:
an ultrasound source configured to generate an ultrasound pulse;
an implant configured to be implanted in body tissue, the implant comprising:
a piezoelectric element configured to receive an ultrasonic pulse and convert the electronic pulse into an electric potential;
a diode coupled to the piezoelectric element, the diode configured to cause an electric current to flow in response to the electric potential;
a reference electrode coupled to the diode, the reference electrode configured to sense bioelectric activity in a region of the body tissue located in proximity to the reference diode; and
a stimulating electrode coupled to the diode, the stimulating diode configured to emit an carrier signal, wherein the carrier signal is modulated in response to the bioelectric activity sensed by the reference electrode; and
a receiver configured to detect the carrier signal.

9. A method comprising:
receiving an ultrasound pulse;
converting the ultrasound pulse into an electric potential;
causing an electric current to flow through a diode from a reference electrode to a stimulating electrode in response to the electric potential; and
emitting an carrier signal from the stimulating electrode, wherein the carrier signal is modulated in response to bioelectric activity in a region of body tissue located in proximity to the reference electrode.

10. The method of claim 9, further comprising:
detecting the carrier signal on a skin surface;
amplifying the carrier signal across a predetermined frequency range;
capturing the carrier signal within a specified time range;
constructing a waveform associated with the carrier signal;
smoothing the waveform; and
producing a waveform display.
